(19) United States
(12) Patent Application Publication
(10) Pub. No.: US 2010/0138234 A1
(43) Pub. Date: Jun. 3, 2010

(54) DEVICE FOR CONDITIONING A MEDICAL APPARATUS OF THE PROBE TYPE

(75) Inventor: Clement Deshays, Ruoms (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: GERMITEC, Clichy (FR)

(21) Appl. No.: 12/594,163

(22) PCT Filed: Apr. 3, 2008
(86) PCT No.: PCT/FR2008/000465

§ 371 Date: Sep. 30, 2009

(30) Foreign Application Priority Data
Apr. 5, 2007 (FR) 0754300

(51) Int. Cl. A61L 2/26; G06Q 10/00; G06Q 50/00; A61B 19/00

(52) U.S. Cl. 705/2; 206/232

(57) ABSTRACT

The invention relates to a device defining an inner housing (5) for receiving at least a portion (2) of the medical apparatus (3), said device including a locking means (7) for closing the housing (5), and said device including a receiving means (9) for identification information (Iᵢ) from the medical apparatus (2) and receiving means for disinfection (l_{d}) and use information from at least one information processing system (D), wherein said locking means (7) are adapted to be activated if the receiving means receives information indicating that the identified medical apparatus (3) to be received has been disinfected, and to prevent any locking if the receiving means receives information indicating that the medical apparatus (3) has not been disinfected.

The present invention relates to a protective packaging device for a probe-type or similar medical instrument, wherein said device forms an internal enclosure intended to house at least a part of the medical instrument, said enclosure being hermetically closable, and said device comprising closure locking means for the enclosure. The invention also relates to a system for tracking the probe-type medical instrument, comprising means for identifying said instrument borne by the medical instrument, at least one information acquisition and processing system comprising means for acquiring the information identifying said instrument, means for acquiring use information regarding the use of said instrument and disinfection information regarding the disinfection of said instrument, date and time recording means, and means for associating the identification, use and disinfection information for said instrument with date and time information.

In the medical field, sanitation of the medical instruments being used is crucial in order to avoid any risk of infecting a patient due to an instrument that has been poorly disinfected or not disinfected. For this reason, it is essential to ensure traceability of the disinfection process in order to be certain that an instrument has in fact been disinfected before it is used.

Currently, this traceability is provided through manual operations by medical personnel. That is, the medical probe is accompanied by a record on which medical personnel must write the disinfection operations that have been carried out, along with the way the probe is used. In some cases, traceability is partially automated, and the information about a probe is assembled in a database. However, in this latter case, medical personnel must still enter the information regarding probe disinfection and use into the database themselves. Consequently, traceability is based on many human actions that can be sources of errors and/or falsification. Therefore, it is impossible to be absolutely certain whether a probe may be legitimately used from the standpoint of hygiene and of preventing nosocomial infections.

The medical probe can be protected between the instrument disinfection and use phases by means of a packaging device that forms a sealed enclosure housing the active part of the probe. However, an operator is who decides when to place the probe in the sealed enclosure and when to take it out so that it can be used. In such an embodiment, there is no verification, other than one involving a person, that the probe has indeed been disinfected before it is placed in the packaging device and that this probe is fit for use before the probe is taken out of the packaging device.

The invention aims to remedy these disadvantages by proposing a packaging device and a tracking system that dispenses with any human involvement in order to guarantee that the probe is legitimately usable from the hygienic standpoint.

To this end, the invention relates to a packaging device of the aforementioned type, wherein said device is characterized in that it comprises means for receiving identification information from the medical instrument, means for receiving disinfection and use information from at least one information processing system, with said locking means being arranged to be activated if the receiving means receive a notice that the identified medical instrument to be housed has been disinfected, and to prevent locking if the receiving means receive a notice that the medical instrument has not been disinfected.

In this way, locking of the packaging enclosure occurs only if the probe has first been disinfected, without making the operator ascertain it himself, since the information is transmitted automatically. If the device prevents the closure from locking, the operator knows that the probe must be disinfected before it is taken to its point of use.

According to other characteristics of the packaging device:
the packaging device comprises means for sending information regarding the medical instrument it contains to the information processing system, with said sending means being arranged to send the information identifying the medical instrument housed in the enclosure to the information processing system when the locking means have locked the enclosure closure;
the locking means are arranged to unlock the enclosure closure when the receiving means receive notice from the information processing system that the medical instrument is going to be used;
the sending means are arranged to inform the information processing system when the enclosure closure is unlocked and the medical instrument is taken out of the enclosure;
the sending means send the identification and disinfection information for the medical instrument that the device contains in its enclosure to the information processing system when the locking means unlocks the enclosure closure;
the packaging device comprises date and time recording means, the locking means being arranged to unlock the enclosure closure when the medical instrument has been present in the enclosure for a predetermined length of time;
the information sending and receiving means are means that operate remotely; and
the information sending and receiving means comprise wire connection means.

The invention also relates to a tracking system of the aforementioned type, wherein said system is characterized in that it comprises a packaging device as defined above, the packaging device housing the medical instrument between the disinfection and use phases of the instrument, wherein the packaging device, the medical instrument, and the information acquisition and processing system are able to communicate with one another in order to transmit information to each other regarding identification of the medical instrument, its disinfection and its use.

Such a tracking system makes it possible to determine with no human intervention whether a probe has been disinfected and may be safely used from the hygienic standpoint; the system is additionally able to prevent said use in cases of non-compliance with the hygiene system. This way, the risk of handling errors is eliminated.

According to other characteristics of the tracking system:
the medical devices with which the probe-type medical instrument must operate, of the disinfection enclosure, storage enclosure, imaging device type, each comprise means for acquiring the identification information, means for acquiring disinfection information and use information, and time and date recording means;
the medical devices with which the probe-type medical instrument must operate, of the disinfection enclosure, storage enclosure, imaging device type, each comprise an information acquisition and processing system; and
the means for acquiring the identification information, the means for acquiring disinfection and use information, and the date and time recording means are assembled in an independent, portable device able to communicate with an information processing system.

Other characteristics and advantages of the invention will appear in the following description, given as an example and with reference to the annexed figures, in which:

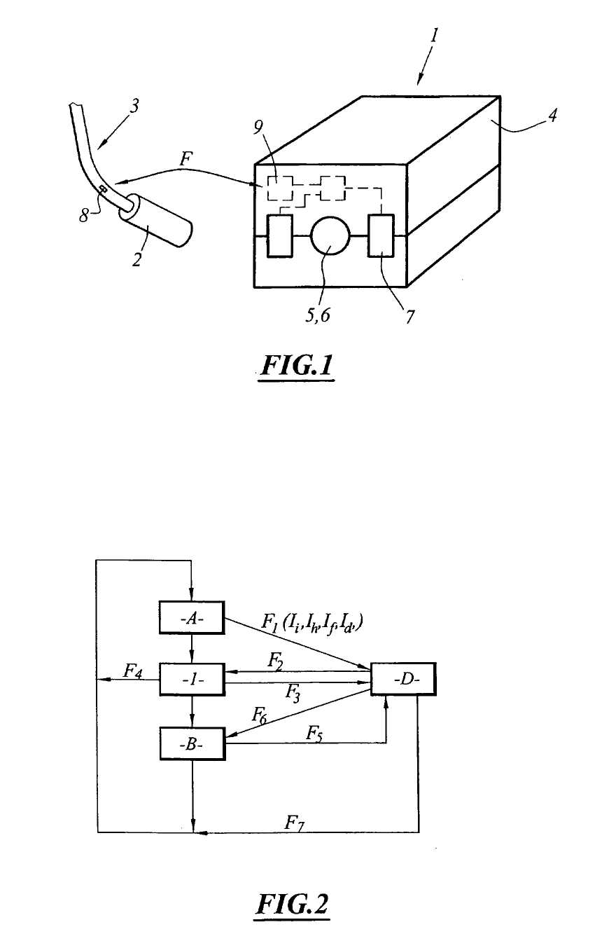

FIG. 1 is a schematic perspective view of a packaging device according to the invention and a probe intended to be placed in the packaging device.

FIG. 2 is a diagram representing the various operating stages of the tracking method according to the invention.

FIG. 1 shows a mobile packaging device 1 intended for housing a part 2 of a probe-type medical instrument 3. The part 2 housed by the packaging device 1 is typically the active part of the probe intended to be in contact with a patient, which must therefore be disinfected so as to avoid any risk of infection.

The mobile packaging device 1 comprises a housing 4 that forms an internal enclosure 5 inside which the part 2 of the probe 3 is intended to be placed, through an opening 6.

The enclosure 5 is designed to be hermetically closed when the part 2 of the probe 3 is placed inside. To this end, the opening 6 is provided with joint, diaphragm, or a similar type of sealing means (not shown).

The housing 4 can be opened in order to insert the part 2 of the probe, and then hermetically closed in order to seal the enclosure 5.

The device comprises closure locking means 7 for the enclosure 5, which make it possible to prevent the enclosure 5 from opening if the locking means are activated. According to an embodiment, the packaging device 1 is autoclavable.

The locking means are electronically controlled so that they are activated to lock the enclosure 5 closure in response to information received by the packaging device 1, as will be described below.

The packaging device 1 is part of a system for tracking the use of a probe 3 that makes it possible to verify whether a probe 3 may be legitimately used from the hygienic standpoint, i.e., whether it has been adequately disinfected, and recently enough, before its use.

The tracking system comprises means 8 for identifying the probe 3 that are borne by the probe 3. For example, these means are an RFID chip permanently fixed on the probe 3, containing information identifying the probe. These identification means can be scanned remotely by means 9 for acquiring the probe identification information. The packaging device 1 comprises the kind of acquisition means 9, e.g., an RFID scanner, that can scan the probe 3 identification information, as shown by the arrow F in FIG. 1. In this way, the packaging device 1 “recognizes” the probe 3 that it contains.

The tracking system additionally comprises at least one information acquisition and processing system D comprising means for acquiring information identifying the probe 3, means for acquiring use information regarding the use of the probe 3 and disinfection information regarding disinfection of the probe 3, date and time recording means, and means for associating probe 3 identification, use, and disinfection information with date and time information. All of these means, which are linked to a database, make it possible to track the disinfection and use of the probe.

Thus, during a stage A shown in FIG. 2, the probe is disinfected. Prior to being disinfected, the probe is identified and the date of disinfection is recorded, along with the time disinfection begins. When disinfection is finished, the disinfection ending time is recorded. The identification information Iᵢ, the date and exam starting time Iₕ information, and the disinfection ending time information I_{f} are transmitted to the central data processing unit D, as shown by the arrow F₁ in FIG. 1. A record is also made of the identity of the operator performing disinfection.

The means for acquiring the identification information and the date and time recording means can be associated with a disinfection enclosure or can be part of an independent, portable device placed in the disinfection room.

According to an embodiment, the disinfection enclosure is provided with identification means so that the identity of the enclosure used for disinfecting the probe is transmitted to the data processing unit D. According to an embodiment, the disinfection enclosure comprises its own information acquisition and processing system which makes it possible to detect the probe disinfection characteristics.

Next, disinfection is performed. Means for acquiring information regarding the use of said instrument make it possible to record the disinfection characteristics. These acquisition means comprise, for example, means for measuring the degree of disinfection provided by a disinfection enclosure. The recorded information can include a record of the active principle used for disinfection, the length of time the instrument was exposed to this active principle, the disinfection temperature and/or the active principle dosage, for example. These measurements can be taken by sensors or by any other known means. This disinfection information I_{d} is transmitted to the data processing unit D, which determines a degree of disinfection from this information. For example, if the active principle dosage is measured, various degrees of disinfection will be reached when the measured dosage exceeds predetermined dosage thresholds. If UV radiation is used for disinfection, the degree of disinfection can be determined from the length of time the probe is exposed to this radiation, combined with the amount of radiation dispensed into the enclosure.

Thus, at the end of stage A, the system for tracking the use of a probe knows the type, degree, and time of disinfection for the probe whose use is being tracked.

The packaging device 1 comprises means for receiving disinfection information, enabling it to use the information processing system D to record the time the probe 3 was disinfected and the type and degree of disinfection for the probe 3, whose identity is known, as shown by the arrow F₂ in FIG. 2.

After disinfection has been completed, the operator places the active part of the probe 3 in the packaging device 1. The packaging device 1 retrieves the information Iᵢ identifying the probe 3, as well as the information regarding its disinfection I_{d}.

If the packaging device receives information that disinfection was indeed performed to an appropriate degree, then the locking of the enclosure 5 closure is actuated electronically, and the active part 2 of the probe 3 is hermetically held in the enclosure 5.

The packaging device 1 is provided with means for sending information regarding the probe that it contains to the information processing system D. These means are arranged so as to inform the information processing system D when the locking means 7 have locked the enclosure closure, as shown by the arrow F₃ in FIG. 2. In this way, the information processing system D can make a list of probes 3 and packaging devices 1 with which these probes 3 are used. When the information processing system D has received notice that a probe 3 is in a packaging device 1, the processing system D communicates the identity of this probe 3 to the other packaging devices 1 so that the latter will not accept a probe with the same identification information. Such an embodiment makes it possible to detect probe identification errors if duplicates have been created erroneously or fraudulently.

If the packaging device retrieves information that disinfection was not performed or that it was performed too much time previously or to an inadequate degree, the enclosure closure is not locked, and the packaging device informs the information processing system D via its sending means that disinfection must take place again, as shown by the arrow F₃ in FIG. 2. The probe is then sent back to the disinfection stage A, as shown by the arrow F₄ in FIG. 2. In addition, the data processing system D sends a notice that the non-disinfected probe 3 cannot be accepted by another packaging device 1. In this way, if an operator tries to use another packaging device 1 to store the probe 3, this device will refuse to lock the enclosure 5 closure as soon as it obtains the information Iᵢ identifying the probe 3.

The packaging device 1 can serve as a storage enclosure for the probe 3 until it is used. According to another embodiment, the probe can be placed with the packaging device 1 in a storage enclosure before being used.

The packaging device 1 comprises date and time recording means able to determine the amount of time a probe 3 has been stored in its enclosure 4. So if this length of time exceeds a predetermined time, the packaging device 1 actuates the unlocking of the enclosure 4 closure and informs the information processing system D that disinfection must take place again. The probe 3 is then sent back to the disinfection stage A and the data processing system D sends a notice that the non-disinfected probe 3 cannot be accepted by another packaging device 1.

During a stage B, the probe 3 is used. Prior to use, the attending physician informs the data processing system D of his or her identity and the upcoming usage conditions for the probe 3. The usage conditions for the probe 3 include the name of the patient, the type of treatment for which the probe 3 is being used, and any other information that is useful for this treatment. To this end, the imaging device with which the probe 3 must be used comprises means for recording the physician's identity, such as an identification card reader, as well as means for entering information regarding the probe usage conditions. The data processing system D verifies whether the probe 3 that must be used can be used legitimately, i.e., whether it has been disinfected appropriately and recently enough. If so, the processing system sends the packaging device 1 a notice to unlock, which then actuates the unlocking of the enclosure 4 closure, as shown by the arrow F₆ in FIG. 2. The physician can then use the probe. According to an embodiment, the disinfection I_{d} probe identification information is transmitted to the imaging device by the data processing system D or the packaging device 1 when a command has been given to unlock the enclosure 5 closure.

The probe 3 is identified and the exam date is recorded. This identification and the date are transmitted to the data processing unit D, as shown by the arrow F₅ in FIG. 1. The processing unit links this information, and a sequencing of disinfection and use is performed. Thus, for a given probe identity, all the information regarding pre-use actions is known and put in chronological order. This way, it can be ensured that a chain of actions has been properly completed before allowing the probe to be used. The probe identity and legitimacy-of-use verification checks can be performed by the imaging device with which the probe is used or by the independent, portable device already used during disinfection and storage. In such a case, the unlock command can be sent by the imaging device itself or by the independent, portable device. If legitimacy of use is not guaranteed for the probe, the packaging device will refuse to unlock and the probe will be sent back to the disinfection stage, as mentioned above. If the independent device is used, it is the latter which comprises means for recording the physician's identity, such as an identification card reader, as well as means for entering information regarding probe usage conditions.

According to an embodiment, the packaging device is unlocked once the name of the patient has been identified in the data processing system D, in the imaging device or in the independent, portable device. Such an embodiment makes it possible to easily associate the probe disinfection characteristics with the patient for whom this probe is used.

The imaging system with which the probe is used can be set up so that it will not operate with a probe for which all the pertinent information has not been transmitted by the packaging device 1.

Once the unlock command has been given for the packaging device 1, the data processing system D sends a notice that the probe 3 can no longer be accepted by another packaging device 1 and must be disinfected again, as shown by the arrow F₇ in FIG. 2. After being used, the probe 3 is sent back to the disinfection stage A. This probe cannot be accepted again by a packaging device 1 until after this stage 1.

The packaging device 1 can communicate with the data processing system(s) D either remotely or by wire connection.

A history tracking the use of the probe is also recorded. This way, after the probe is used by a physician, the data processing unit D can produce specific disinfection instructions (degree and type of disinfection) depending on the type of treatment for which the probe was used.

According to an embodiment, all the information transmitted to the packaging device 1 is also transmitted to the probe itself, which comprises means for storing this information. In this way, the probe can have the capabilities described above for analyzing the legitimacy of its use.

The invention thus makes it possible to guarantee that a particular probe is always used legitimately from the standpoint of hygiene, and to prevent a dangerous use of this probe.

1. Mobile packaging device for a probe-type or similar medical instrument, wherein said device forms an internal enclosure intended to house at least a part (2) of the medical instrument, said enclosure being hermetically closable, and said device comprising:
closure locking means for the enclosure (5),
means for receiving identification information (lᵢ) from the medical instrument, and
means for receiving disinfection and use information from at least one information processing system,
said locking means being arranged to be activated if the receiving means receive a notice that the identified medical instrument to be housed has been disinfected, and to prevent locking if the receiving means receive a notice that the medical instrument has not been disinfected.

2. Packaging device according to claim 1, which comprises means for sending information regarding the medical instrument it contains to the information processing system, with said sending means being arranged to send the information identifying the medical instrument housed in the enclosure to the information processing system when the locking means have locked the enclosure closure.

3. Packaging device according to claim 1, wherein the locking means are arranged to unlock the enclosure closure when the receiving means receive notice from the information processing system that the medical instrument is going to be used.

4. Packaging device according to claim 3, wherein the sending means are arranged to inform the information processing system when the enclosure closure is unlocked and the medical instrument is taken out of the enclosure.

5. Packaging device according to claim 4, wherein the sending means send the identification and disinfection information for the medical instrument that the device contains in its enclosure to the information processing system when the locking means unlock the enclosure closure.

6. Packaging device according to claim 1, which comprises date and time recording means, the locking means being arranged to unlock the enclosure closure when the medical instrument has been present in the enclosure for a predetermined length of time.

7. Packaging device according to claim 2, wherein the information sending and receiving means are means that operate remotely.

8. Packaging device according to claim 2, wherein the information sending and receiving means comprise wire connection means.

9. Packaging device according to claim 1, wherein the packaging device is autoclavable.

10. System for tracking the use of a probe-type medical instrument, comprising:
means for identifying said instrument borne by the medical instrument,
at least one information acquisition and processing system comprising means for acquiring information identifying said instrument, means for acquiring use information regarding the use of said instrument and disinfection information regarding the disinfection of said instrument, date and time recording means, and means for associating the identification, use and disinfection information for said instrument with date and time information,
wherein said system comprises a packaging device according to claim 1,
wherein the packaging device houses the medical instrument between the disinfection and use phases of the instrument, and the packaging device, the medical instrument and the information acquisition and processing system are able to communicate with one another in order to transmit information to each other regarding identification of the medical instrument, its disinfection and its use.

11. Tracking system according to claim 10, wherein medical devices with which the probe-type medical instrument must operate, of the disinfection enclosure, storage enclosure, imaging device type, each comprise means for acquiring identification information, means for acquiring disinfection information and use information, and time and date recording means.

12. Tracking system according to claim 10, wherein the medical devices with which the probe-type medical instrument must operate, of the disinfection enclosure, storage enclosure, imaging device type, each comprise an information acquisition and processing system.

13. Tracking system according to claim 10, wherein the means for acquiring identification information, the means for acquiring disinfection and use information, and the date and time recording means are assembled in an independent, portable device able to communicate with an information processing system.
